(19) United States
(12) Patent Application Publication
(10) Pub. No.: US 2006/0264671 A1
(43) Pub. Date: Nov. 23, 2006

(54) Process for the preparation of midodrine, pharmaceutically acceptable salts thereof and intermediates

(76) Inventors: Gamini Weeratunga, Brantford (CA); Elena Bejan, Brantford (CA)

Correspondence Address: IVOR M. HUGHES, BARRISTER & SOLICITOR,;PATENT & TRADEMARK AGENTS, 175 COMMERCE VALLEY DRIVE WEST, SUITE 200, THORNHILL, ON, L3T 7P6, CA

(21) Appl. No.: 10/547,538

(22) PCT Filed: Mar. 10, 2004
(86) PCT No.: PCT/CA04/00347

§ 371 Date: Jun. 16, 2006

(30) Foreign Application Priority Data
Mar. 11, 2003 (CA) 2,421,550

(51) Int. Cl. C07C 237/20

(52) U.S. Cl. 564/133; 564/194

(57) ABSTRACT

The present invention provides for a novel process for the preparation of Midodrine or a pharmaceutically acceptable salt thereof comprising: (a) a step of reacting 2-amino-1-(2′,5′-dimethoxyphenyl) ethanol of formula (I) with an N-protected glycine of formula (II) containing an amino protecting group in the presence of 1,1′-carbonyldiimidazole (CDI); and (b) removing the amino protecting group by deprotection formula (I), formula (II), wherein R₁ is a benzyl, triphenylmethyl, tert-butyloxycarbonyl, or a benzyloxycarbonyl group. This results in an unexpectedly efficient and cost-effective process. Additionally, the process is simple and safe as all the intermediates and reagents involved in the process pose no safety risks. Further reaction of Midodrine with a pharmaceutically acceptable acid affords a pharmaceutically acceptable salt thereof. Preferably, the pharmaceutically acceptable salt obtained from the process according to the present invention is Midodrine Hydrochloride.

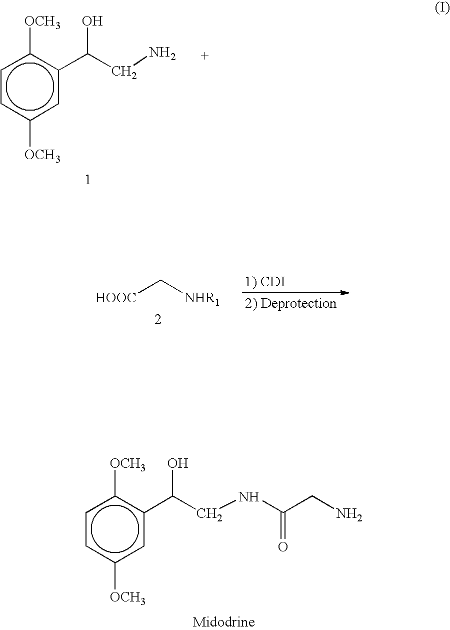

### FIELD OF INVENTION

The present invention refers to a new process for the synthesis of Midodrine, pharmaceutically-acceptable salts thereof and intermediates.

### BACKGROUND OF THE INVENTION

Midodrine Hydrochloride is a phenylalkanolamine derivative marketed as an effective antihypotensive drug. It was first described in the U.S. Pat. No. 3,340,298 (U.S. Pat. No. '298). The method of preparation taught in U.S. Pat. No. '298 is based on a conventional amidation reaction in which the aminoethanol derivatives of formula 3 are reacted with protected aminoacids or aminoacid derivatives of formula 4 in the presence of N,N′-dicydohexylcarbodiimide (DCC) to form an amide bond (Scheme 1). The obtained intermediates of formula 5 are then deprotected by hydrogenation under pressure in acetic acid to yield after treatment with hydrochloric acid, Midodrine Hydrochloride in very low overall yields of 30-40%.

A serious drawback of the described method of synthesis identified by Bodanszky [M. Bodanszky, Principles of Peptide Synthesis, Springer Verlag 1993, page 40] is that the major by-product, N,N′-dicyclohexylurea (DCU), while indeed insoluble in most organic solvents, is not entirely insoluble and it frequently becomes trapped and contaminates the coupling product. Therefore, the isolated intermediate of formula 5 has to be purified before utilization in the deprotection step.

Also noteworthy is that DCU is highly toxic and DCC is allergenic and, therefore, these chemicals present handling issues in a commercial manufacturing environment.

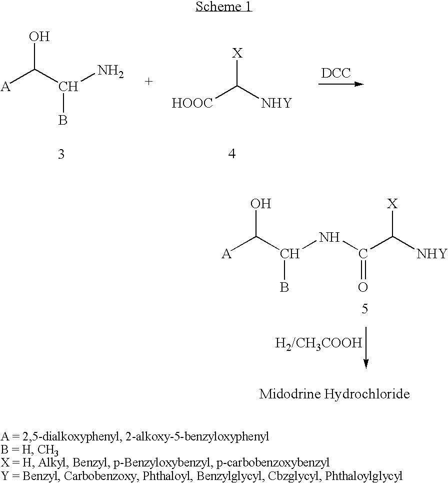

U.S. Pat. No. 6,201,153 purportedly overcomes the DCU deficiency encountered in U.S. Pat. No. '298 by using as intermediate the anhydride of formula 6. The synthetic scheme described in U.S. Pat. No. 6,201,153 involves a three step procedure with an overall yield of 69%. In the first step, the anhydride of formula 6 is prepared by reacting N-Boc-glycine with DCC in a 2:1 ratio, in dry dichloromethane (Scheme 2). The DCU by-product thus formed is precipitated out at 0° C. to provide the anhydride of formula 6 in solution.

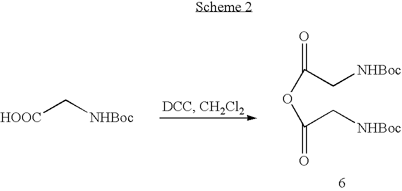

The produced anhydride of formula 6 is then reacted with the 2-amino-1-(2′,5′-dimethoxyphenyl)-ethanol of formula 1 in the presence of 4 dimethylaminopyridine (DMAP) in dichloromethane to provide the N-tert-butoxycarbonyl Midodrine intermediate of formula 7 in an 80% yield. In the final step, Midodrine Hydrochloride is obtained by deprotection of the N-tert-butoxycarbonyl Midodrine intermediate of formula 7 with concentrated HCl/acetone, HCl gas/MeOH or concentrated HCl-AcOEt in an 87% yield (Scheme 3).

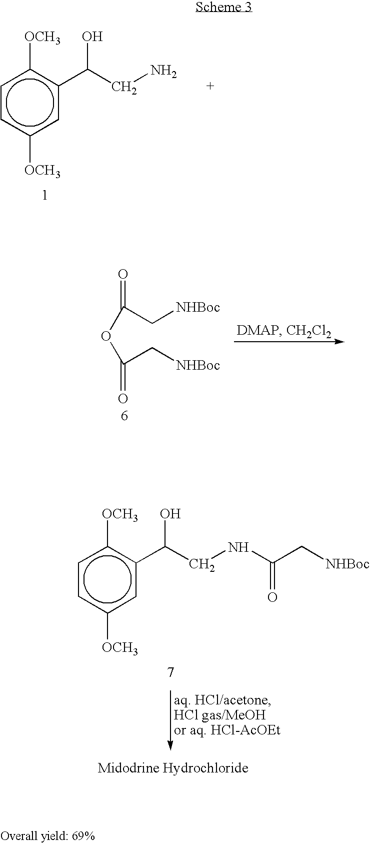

Thus the described method of synthesis involves a three step procedure with an overall yield of 69%. The main shortcoming of the described procedure is the formation of the highly toxic and difficult to remove DCU by-product as previously mentioned in U.S. Pat. No. '298. Another drawback of this method is the utilization of highly toxic and allergenic reagents (dichloromethane, DCC) in the preparation of anhydride of formula 6 and intermediate of formula 7.

### SUMMARY OF THE INVENTION

The drawbacks linked to the prior art processes are overcome by a novel process for the synthesis of Midodrine and pharmaceutically-acceptable salts thereof, based on a single reactor and commercially viable procedure involving the coupling of the 2-amino-1-(2′,5′-dimethoxyphenyl) ethanol of formula 1 with N-protected glycines of formula 2 using a safe and convenient coupling reagent, followed by the in situ deprotection of N-protected Midodrine intermediates.

An object of the present invention is to provide a new and improved process for the preparation of Midodrine or a pharmaceutically acceptable salt thereof comprising:
(a) reacting 2-amnion-1-(2′,5′-dimethoxyphenyl) ethanol of formula 1 with an N-protected glycine of formula 2 containing an amino protecting group preferably in the presence of 1,1′-carbonyldiimidazole (CDI); and
(b) removing the amino protecting group by deprotection 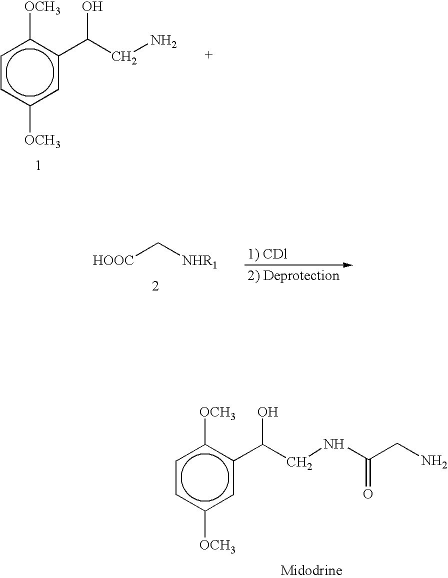
wherein R₁ is a benzyl, triphenylmethyl, tert-butyloxycarbonyl, or a benzyloxycarbonyl group.

Another object of the present invention provides for the reaction of Midodrine with an acid to afford a pharmaceutically acceptable salt thereof.

Another object of the present invention is to provide a process for the preparation of the pharmaceutically acceptable salt Midodrine Hydrochloride.

According to one aspect of the present invention, the process for the preparation of Midodrine or a pharmaceutically acceptable salt thereof is carried out when CDI is preferably in an organic solvent. Even more preferably, the organic solvent is selected from the group consisting of C₂-C₄ nitrile solvents, C₂-C₇ ester solvents and C₁-C₄ amide solvents and mixtures thereof. Preferably, the organic solvent is selected from the group consisting of ethyl acetate, acetonitrile, dimethylformamide and mixtures thereof.

In one aspect of the process according to the invention, the removal of the amino protecting group (deprotection) preferably comprises:
(a) a reaction with HCl; or
(b) a hydrogenation reaction.

In a preferred embodiment of the invention, the process further comprises an addition of HCl after the hydrogenation reaction to yield Midodrine Hydrochloride.

Preferably, the hydrogenation reaction is either a hydrogenation under pressure or a catalytic transfer hydrogenation.

Even more preferably, the catalytic transfer hydrogenation is carried out in the presence of at least one catalytic transfer agent, preferably said at least one catalytic transfer agent is selected from the group consisting of cyclohexene, 1,4-cydohexadiene, formic acid, ammonium formate, hydrazine and mixtures thereof.

Preferably the hydrogenation reaction is carried out in the presence of at least one catalyst, preferably Pd/C or Pd black as catalyst. Even more preferably, the hydrogenation reaction is carried out in the presence of a solvent selected from the group consisting of methanol, ethanol, acetic acid and a mixture of acetic acid/ethanol. In a preferred embodiment of the present invention, the hydrogenation reaction is carried out under a hydrogen pressure of about 40 to about 100 psi. Preferably, the hydrogenation reaction is carried out at a temperature of about 40° C. to about 70° C.

According to another aspect of the invention, the hydrogenation reaction is followed by the addition of hydrochloric acid to yield the hydrochloride salt of Midodrine.

According to yet another aspect of the invention, the deprotection is carried out using hydrochloric acid at a temperature ranging from about 20° C. to about 50° C.

Preferably, the deprotection is carried out using hydrochloric acid in isopropanol, preferably anhydrous hydrochloric acid is used.

According to another aspect of the present invention, there is provided a process for the preparation of N-protected Midodrine intermediates of formula 8 by reacting 2-amino-1-(2′,5′-dimethoxyphenyl)-ethanol of formula 1 with an N-protected glycine of formula 2 in the presence of 1,1′-carbonyldiimidazole (CDI); 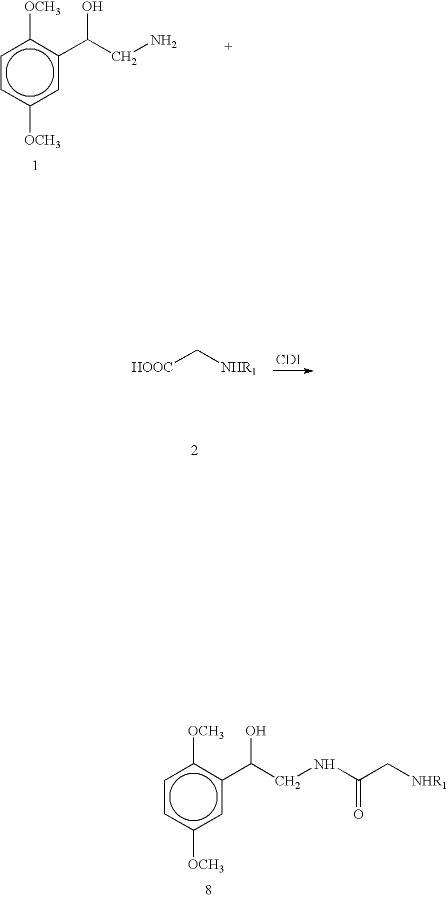
wherein R₁ is a benzyl, triphenylmethyl, tert-butyloxycarbonyl, or a benzyloxycarbonyl group.

Preferably, in the process, CDI is in an organic solvent preferably selected from the group consisting of C₂-C₄ nitrile solvents, C₂-C₇ ester solvents and C₁-C₄ amide solvents or mixtures thereof. More preferably, the organic solvent is selected from the group consisting of ethyl acetate, acetonitrile, dimethylformamide and mixtures thereof.

Yet another object of the present invention provides for a process for the preparation of Midodrine hydrochloride comprising:
(a) reacting 2-amino-1-(2′,5′-dimethoxyphenyl) ethanol of formula 1 with an N-protected glycine of formula 2 containing an amino protecting group in the presence of 1,1′-carbonyldiimidazole (CDI) and in an organic solvent selected from a group consisting of ethyl acetate, acetonitrile, dimethylformamide and mixtures thereof; and
(b) removing the amino protecting group and formation of the Hydrochloride salt by addition of HCl 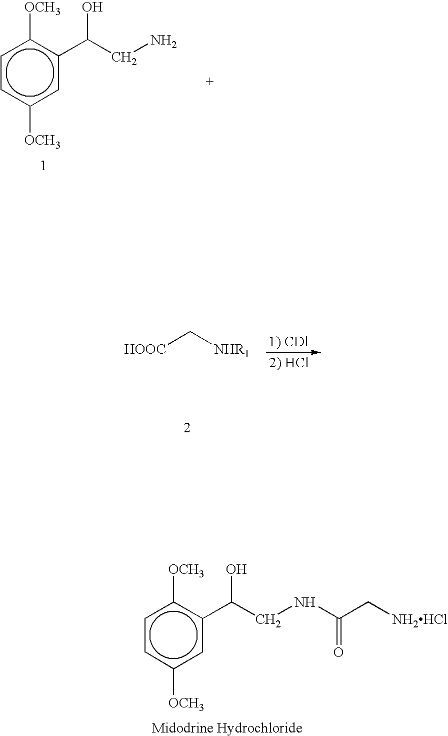
wherein R₁ is a triphenylmethyl or a tert-butyloxycarbonyl group.

Some of the advantages of the current process include a substantially high yield, in one instance about 87%, substantially fewer steps, a cost effective process and a substantially higher productivity by carrying out more than one synthetic transformation in one reactor. Additionally, the proposed invention is simple and safe, as all the reagents and intermediates involved in the process pose no safety risks. Relative to the prior art, this process efficiently provides Midodrine and pharmaceutically acceptable salts thereof, in one instance the Hydrochloride salt in substantially high yield based on the present invention. Further advantages associated with the present invention will be readily seen in reviewing the detailed description of the invention.

Further and other objectives of the present invention will be readily understood in reviewing the following detailed description of the invention.

### DETAILED DESCRIPTION OF THE INVENTION

In a preferred embodiment of the present invention, the process relates to the preparation of Midodrine Hydrochloride, also known as (±)-2-amino-N-[2-(2′,5′-dimethoxyphenyl)-2-hydroxyethyl)acetamide hydrochloride.

The 2-amino-1-(2′,5′-dimethoxyphenyl) ethanol of formula 1, was prepared starting from 2,5-dimethoxybenzene by using processes described in the literature [E. Epifani, A. Lapucci, B. Macchia, F. Macchia, P. Tognetti, M. C. Breschi, M. Del Tacca, E. Martinotti, L. Giovanninni, J. Med. Chem. 1983, 26, pages 254-259].

The novel process according to the present invention is based on a single reactor and easy to scale-up procedure involving the coupling of the 2-amino-1-(2′,5′-dimethoxyphenyl) ethanol of formula 1 with commercially available N-protected glycines of formula 2, followed by the in situ deprotection of N-protected Midodrine intermediates.

This novel process according to the present invention consists of reacting 2-amino-1-(2′,5′-dimethoxyphenyl) ethanol of formula 1 with an N-protected glycines of formula 2 in ethyl acetate and in the presence of 1,1′-carbonyldiimidazole (CDI) to yield the N-protected Midodrine intermediates (Scheme 4).

The by-products of the CDI coupling are CO₂ and imidazole, which are considerably less toxic than DCU. 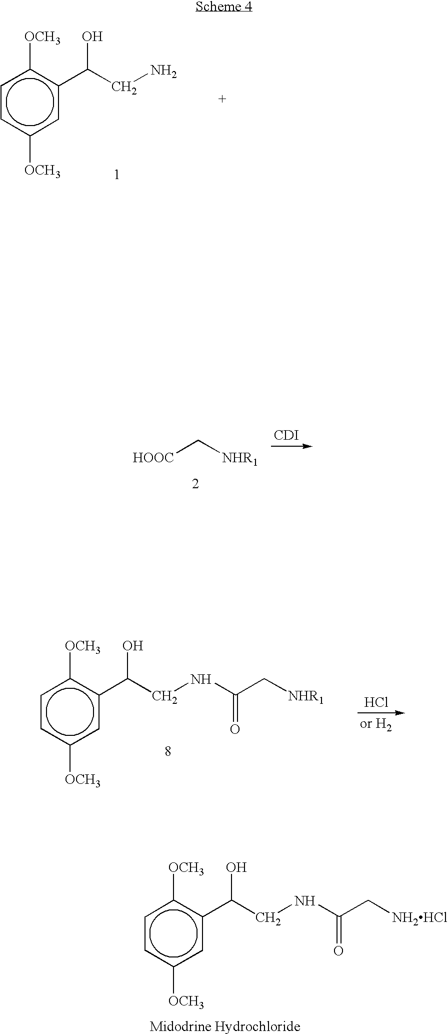
wherein R₁ is a benzyl, triphenylmethyl, tert-butyloxycarbonyl, benzyloxycarbonyl group.

The N-protected Midodrine intermediates of formula 8 when R₁=triphenylmethyl or a tert-butyloxycarbonyl are not isolated but reacted, after work-up, with aqueous HCl to yield, after filtration, Midodrine Hydrochloride in substantially highly pure form and a yield of 87% (HPLC purity: 98.1%).

When R₁=benzyl or benzyloxycarbonyl, the N-protected Midodrine intermediates are not isolated but hydrogenated at about 60 psi and about 60° C. in acetic acid, ethanol, methanol or mixtures of acetic acid/ethanol, in the presence of Pd/C or Pd black as catalyst to yield after precipitation with hydrochloric acid highly pure form of Midodrine Hydrochloride in excellent yield.

It should also be noted that all the reagents and intermediates used in the described process as well as the reaction by-products pose no safety risks.

The following examples are merely illustrative and are not intended to limit the scope of the present invention in any manner. Although, the examples described in the patent are all based on 50 g scale experiments, kilogram scale batches have been conducted successfully.

### EXAMPLE 1

Preparation of Midodrine Hydrochloride

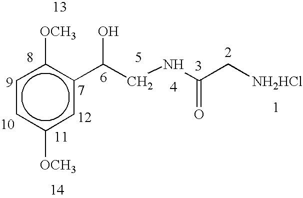

1,1′-Carbonyldiimidazole (45.32 g, 0.279 moles) was suspended in ethyl acetate (100 ml). To the beige suspension is added portionwise N-tert-butoxycarbonyl glycine (48.95 g, 0.279 moles). After stirring for 1 hour, this solution was added to a suspension of 2-amino-1-(2′,5′-dimethoxyphenyl) ethanol (50.0 g, 0.253 moles) in ethyl acetate (250 ml). The reaction mixture is stirred at room temperature for 1 hour. A solution of 8% hydrochloric acid (220 ml, 2.2 equiv.) is added to the reaction mixture and the mixture stirred at room temperature for 15 minutes. Stirring is discontinued and the phases are separated. The organic layer is sequentially washed with water, sodium hydroxide 2.5% and water and then dried over sodium sulfate. To the clear ethyl acetate solution is added hydrochloric acid 32% (76 ml, 3 equiv.) and the white suspension stirred at room temperature for 4 hours. The white solid (Midodrine Hydrochloride: 63.9 g, 87%) was filtered, washed with ethyl acetate and dried. HPLC purity is found to be 98.1%. The product is characterized as follows:

LRMS(ESI): 255.33 (100, [M-HCl+H]⁺).

EA: C 49.63% (calc. 49.53); H 6.53% (calc. 6.59); N 9.55% (calc. 9.64).

¹H NMR (DMSO-d₆): δ (ppm) 8.58 (1H, t, J=5.3 Hz, He); 8.28 (3H, s, H₁); 7.04 (1H, d, J=2.9 Hz, H₁₂); 6.90 (1H, d, J=8.9 Hz, Hg); 6.79 (1H, dd, J=2.9, 8.8 Hz, H₁₀); 5.54 (10H, d, J=4.3 Hz); 5.0-4.88 (1H, m, H₆); 3.74 (3H, s, H₁₃); 3.71 (3H, s, H₁₄); 3.56 (1H, ad, H₂ₐ); 3.47 (1H, ad, H_{2b}); 3.50-3.38 (1H, m, H_{1b}); 3.12-3.00 (1H, m, H₅ₐ).

¹³C NMR (DMSO-d₆): δ (ppm) 165.8, C₃; 153.2, C₁₁; 149.7, C₈; 132.3, C₇; 112.7, C₁₂; 112.3, C₁₀; 111.7, C₉; 65.5, C₆; 55.9, C₁₃; 55.3, C₁₄; 45.6, C₅; 40.1, C₂.

### EXAMPLE 2

Preparation of Midodrine Hydrochloride

1,1′-Carbonyldiimidazole (45.32 g, 0.279 moles) was suspended in ethyl acetate (100 ml). To the beige suspension is added portionwise Carbobenzyloxyglycine (58.36 g, 0.279 moles). After stirring for 1 hour, this solution was added to a suspension of 2-amino-1-(2′,5′-dimethoxyphenyl) ethanol (50.0 g, 0.253 moles) in ethyl acetate (250 ml). The reaction mixture is stirred at room temperature for 1 hour. A solution of 8% hydrochloric acid (220 ml, 2.2 equiv.) is added to the reaction mixture and the mixture stirred at room temperature for 15 minutes. Stirring is discontinued and the phases are separated. The organic layer is sequentially washed with water, sodium hydroxide 2.5% and water and then dried over sodium sulfate. After distillation of the majority of the ethyl acetate layer, to the solution is added 400 ml of acetic acid and 7.5 g 5% Pd/C. The suspension is then hydrogenated at 60 psi and 60° C. for 24 hours. On reaction completion, the mixture is cooled to 22-26° C. and filtered through Celite. The clear filtrate is evaporated to 300 ml and hydrochloric acid 32% (25 ml, 1 equiv.) is added dropwise to afford Midodrine Hydrochloride.

1. A process for the preparation of Midodrine or a pharmaceutically acceptable salt thereof comprising:
(a) reacting 2-amino-1-(2′,5′-dimethoxyphenyl) ethanol of formula 1 with an N-protected glycine of formula 2 containing an amino protecting group in the presence of 1,1′-carbonyldiimidazole (CDI); and
(b) removing the amino protecting group by deprotection 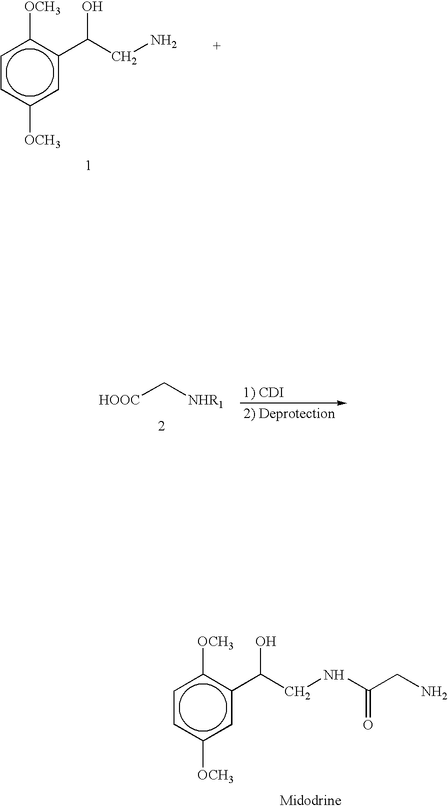
wherein R₁ is a benzyl, triphenylmethyl, tert-butyloxycarbonyl, or a benzyloxycarbonyl group.

2. The process according to claim 1 wherein Midodrine is further reacted with an acid to afford a pharmaceutically acceptable salt thereof.

3. The process according to claim 1 or 2 wherein the pharmaceutically acceptable salt is Midodrine Hydrochloride.

4. The process according to claim 1 or 2 wherein CDI is in an organic solvent and the organic solvent is selected from the group consisting of C₂-C₄ nitrile solvents, C₂-C₇ ester solvents, C₁-C₄ amide solvents and mixtures thereof.

5. The process according to claim 3 wherein CDI is in an organic solvent and the organic solvent is selected from the group consisting of C₂-C₄ nitrile solvents, C₂-C₇ ester solvents, C₁-C₄ amide solvents and mixtures thereof.

6. The process according to claim 4 wherein the organic solvent is selected from the group consisting of ethyl acetate, acetonitrile, dimethylformamide and mixtures thereof.

7. The process according to claim 5 wherein the organic solvent is selected from the group consisting of ethyl acetate, acetonitrile, dimethylformamide and mixtures thereof.

8. The process according to any one of claims 1, 2, 5, 6 or 7 wherein the deprotection comprises:
(a) a reaction with HCl; or
(b) a hydrogenation reaction.

9. The process according to claim 3 wherein the deprotection comprises:
(a) a reaction with HCl; or
(b) a hydrogenation reaction.

10. The process according to claim 4 wherein the deprotection comprises:
(a) a reaction with HCl; or
(b) a hydrogenation reaction.

11. The process according to claim 8 further comprising addition of HCl after the hydrogenation reaction to yield Midodrine Hydrochloride.

12. The process according to claim 9 or 10 further comprising addition of HCl after the hydrogenation reaction to yield Midodrine Hydrochloride.

13. The process according to claim 8 wherein the hydrogenation reaction is either a hydrogenation under pressure or a catalytic transfer hydrogenation.

14. The process according to claim 9 or 10 wherein the hydrogenation reaction is either a hydrogenation under pressure or a catalytic transfer hydrogenation.

15. The process according to claim 13 wherein the catalytic transfer hydrogenation is carried out in the presence of at least one catalytic transfer agent selected from the group consisting of cyclohexene, 1,4-cyclohexadiene, formic acid, ammonium formate, hydrazine and mixtures thereof.

16. The process according to claim 14 wherein the catalytic transfer hydrogenation is carried out in the presence of at least one catalytic transfer agent selected from the group consisting of cyclohexene, 1,4-cyclohexadiene, formic acid, ammonium formate, hydrazine and mixtures thereof.

17. The process according to claim 8 wherein the hydrogenation reaction is carried out in the presence of Pd/C or Pd black as catalyst.

18. The process according to any one of claims 9, 10, 11, 13, 15 or 16 wherein the hydrogenation reaction is carried out in the presence of Pd/C or Pd black as catalyst.

19. The process according to claim 12 wherein the hydrogenation reaction is carried out in the presence of Pd/C or Pd black as catalyst.

20. The process according to claim 14 wherein the hydrogenation reaction is carried out in the presence of Pd/C or Pd black as catalyst.

21. The process according to claim 8 wherein the hydrogenation reaction is carried out in the presence of a solvent selected from the group consisting of methanol, ethanol, acetic acid and a mixture of acetic acid/ethanol.

22. The process according to any one of claims 9, 10, 11, 13, 15, 16, 17, 19 or 20 wherein the hydrogenation reaction is carried out in the presence of a solvent selected from the group consisting of methanol, ethanol, acetic acid and a mixture of acetic acid/ethanol.

23. The process according to claim 12 wherein the hydrogenation reaction is carried out in the presence of a solvent selected from the group consisting of methanol, ethanol, acetic acid and a mixture of acetic acid/ethanol.

24. The process according to claim 14 wherein the hydrogenation reaction is carried out in the presence of a solvent selected from the group consisting of methanol, ethanol, acetic acid and a mixture of acetic acid/ethanol.

25. The process according to claim 18 wherein the hydrogenation reaction is carried out in the presence of a solvent selected from the group consisting of methanol, ethanol, acetic acid and a mixture of acetic acid/ethanol.

26. The process according to claim 8 wherein the hydrogenation reaction is carried out under a hydrogen pressure of about 40 to about 100 psi.

27. The process according to any one of claims 9, 10, 11, 13, 15, 16, 17, 19, 20, 21, 23, 24 or 25 wherein the hydrogenation reaction is carried out under a hydrogen pressure of about 40 to about 100 psi.

28. The process according to claim 12 wherein the hydrogenation reaction is carried out under a hydrogen pressure of about 40 to about 100 psi.

29. The process according to claim 14 wherein the hydrogenation reaction is carried out under a hydrogen pressure of about 40 to about 100 psi.

30. The process according to claim 18 wherein the hydrogenation reaction is carried out under a hydrogen pressure of about 40 to about 100 psi.

31. The process according to claim 22 wherein the hydrogenation reaction is carried out under a hydrogen pressure of about 40 to about 100 psi.

32. The process according to claim 8 wherein the hydrogenation reaction is carried out at a temperature of about 40° C. to about 70° C.

33. The process according to any one of claims 9, 10, 11, 13, 15, 16, 17, 19, 20, 21, 23, 24, 25, 26, 28, 29, 30 or 31 wherein the hydrogenation reaction is carried out at a temperature of about 40° C. to about 70° C.

34. The process according to claim 12 wherein the hydrogenation reaction is carried out at a temperature of about 40° C. to about 70° C.

35. The process according to claim 14 wherein the hydrogenation reaction is carried out at a temperature of about 40° C. to about 70° C.

36. The process according to claim 18 wherein the hydrogenation reaction is carried out at a temperature of about 40° C. to about 70° C.

37. The process according to claim 22 wherein the hydrogenation reaction is carried out at a temperature of about 40° C. to about 70° C.

38. The process according to claim 27 wherein the hydrogenation reaction is carried out at a temperature of about 40° C. to about 70° C.

39. The process according to any one of claims 1, 2, 5, 6, 7, 9 or 10 wherein the deprotection is carried out using hydrochloric acid at a temperature ranging from about 20° C. to about 50° C.

40. The process according to claim 3 wherein the deprotection is carried out using hydrochloric acid at a temperature ranging from about 20° C. to about 50° C.

41. The process according to claim 4 wherein the deprotection is carried out using hydrochloric acid at a temperature ranging from about 20° C. to about 50° C.

42. The process according to claim 8 wherein the deprotection is carried out using hydrochloric acid at a temperature ranging from about 20° C. to about 50° C.

43. The process according to any one of claims 1, 2, 5, 6, 7, 9, 10, 40, 41 or 42 wherein the deprotection is carried out using hydrochloric acid in isopropanol.

44. The process according to claim 3 wherein the deprotection is carried out using hydrochloric acid in isopropanol.

45. The process according to claim 4 wherein the deprotection is carried out using hydrochloric acid in isopropanol.

46. The process according to claim 8 wherein the deprotection is carried out using hydrochloric acid in isopropanol.

47. The process according to claim 39 wherein the deprotection is carried out using hydrochloric acid in isopropanol.

48. A process for the preparation of N-protected Midodrine intermediates of formula 8 by reacting 2-amino-1-(2′,5′-dimethoxyphenyl)-ethanol of formula 1 with an N-protected glycine of formula 2 in the presence of 1,1′-carbonyldiimidazole (CDI) 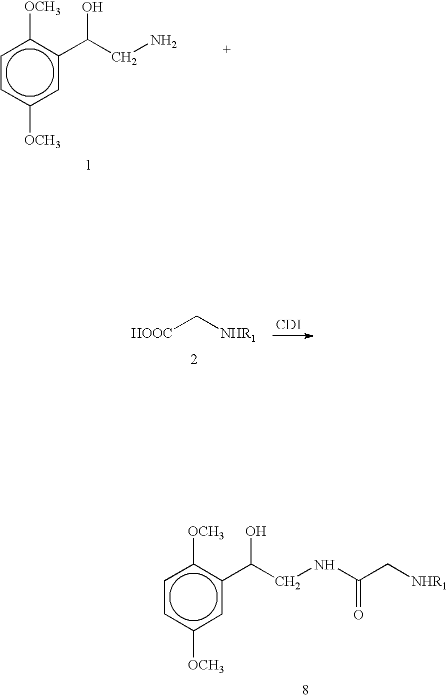
wherein R₁ is a benzyl, triphenylmethyl, tert-butyloxycarbonyl, or a benzyloxycarbonyl group.

49. The process according to claim 48 wherein CDI is in an organic solvent selected from a group consisting of C₂-C₄ nitrile solvents, C₂-C₇ ester solvents, C₁-C₄ amide solvents and mixtures thereof.

50. The process according to claim 49 wherein the organic solvent is selected from the group consisting of ethyl acetate, acetonitrile, dimethylformamide and mixtures thereof.

51. A process for the preparation of Midodrine Hydrochloride comprising:
(a) reacting 2-amino-1-(2′,5′-dimethoxyphenyl) ethanol of formula 1 with an N-protected glycine of formula 2 containing an amino protecting group in the presence of 1,1′-carbonyldiimidazole (CDI) and in an organic solvent selected from a group consisting of ethyl acetate, acetonitrile and dimethylformamide, or mixtures thereof; and
(b) removing the amino protecting group and formation of the Hydrochloride salt by addition of HCl 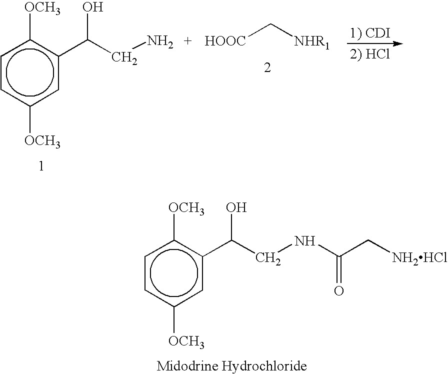
wherein R₁ is a benzyl, triphenylmethyl, tert-butyloxycarbonyl, or a benzyloxycarbonyl group.
